(19) United States
(12) Patent Application Publication
(10) Pub. No.: US 2006/0247187 A1
(43) Pub. Date: Nov. 2, 2006

(54) Attenuation of disorders by aminoglycosides

(76) Inventor: Merlin Thomas, Melbourne (AU)

Correspondence Address: Edwin V. Merkel;Nixon Peabody LLP, Clinton Square, P.O. Box 31051, Rochester, NY, 14603-1051, US

(21) Appl. No.: 11/115,440

(22) Filed: Apr. 27, 2005

(51) Int. Cl. A61K 31/7034; A61K 31/155

(52) U.S. Cl. 514/35; 514/36; 514/37; 514/38; 514/39; 514/634

(57) ABSTRACT

The present invention provides a method of attenuating side effects caused by aminoglycosides by administering aminoguanidine either before or during the treatment with the aminoglycoside. The invention also provides compositions and kits for use in the treatment of infections that use aminoglycosides that can cause side effects such as nephrotoxicity and ototoxicity.

### FIELD OF INVENTION

The present invention relates to methods that attenuate or reduce side effects caused by aminoglycosides. The invention also provides compositions and kits for use in the treatment of infections that utilise aminoglycosides that can cause side effects such as nephrotoxicity and ototoxicity.

### BACKGROUND OF THE INVENTION

Aminoglycosides are widely used and inexpensive antibiotic agents employed in the treatment of infection by gram-negative bacteria, including Pseudomonas, Enterobacter, Proteus, and Neisseria species. Because of their effectiveness and the low rate of true resistance, aminoglycosides are often considered the drug of choice to treat life-threatening infections, such as bacterial endocarditis, peritonitis, and sepsis. They also have synergy with other antibacterial classes (e.g., β-lactams) which frequently proves to be important in the management of chronic or recalcitrant infections.

There are several aminoglycoside antibiotics that are currently in clinical use. Gentamicin is the most commonly used aminoglycoside antibiotic, derived from Microspora purprae. Other aminoglycvoside antibiotics include amikacin, gentamicin, kanamycin, neomycin, netilmicin, paromomycin, streptomycin, and tobramycin. All of these drugs have the same basic chemical structure and a similar range of toxic side-effects.

Aminoglycoside antibiotics are one of the most common causes of drug-induced nephrotoxicity and renal impairment, accounting for up to 15% of cases of iatrogenic acute renal failure. In prospective studies, up to 50% of all patients using gentamicin for over 7 days show signs of nephrotoxicity. The advent of more effective drug monitoring procedures, including better recognition of risk factors and the introduction of once-a-day treatment schedules, has reduced risk of irreversible renal damage, but nephrotoxicity nevertheless continues to represent a considerable limitation to gentamicin use. The requirement for elaborate dosing and monitoring procedures, implemented to maintain doses within a narrow therapeutic range, results in a significant increase in costs of therapy. This may be particularly burdensome in the setting of severe infections, where aminoglycosides can otherwise be life-saving.

Several risk factors for aminoglycoside nephrotoxicity have been identified. The risk of toxicity may be increased in advanced age because of decreased renal function and the reduced regenerative capacity of a damaged kidney. Preexisting renal disease can expose patients to drug accumulation. Hypomagnesemia, hypokalemia, and calcium deficiency may be predisposing risk factors for aminoglycoside-induced renal injury. Other risk factors include prolonged dosing, or high cumulative daily dosing or recent aminoglycoside therapy. Concomitant use of nephrotoxic drugs (e.g., NSAIDS, cephalosporins, frusemide) and calcium channel agents may also increase the risk of renal damage.

Despite the risks of nephrotoxicity arising from aminoglycoside usage, for some infections, the aminoglycoside gentamicin is the only option, and gentamicin toxicity is unavoidable particularly with prolonged use. At present the only ways to avoid gentamicin toxicity are to (a) avoid gentamicin altogether for a more expensive less potent compound and (b) reduce the dose and or length of treatment (which may compromise efficacy in some settings)

Hence a more effective way of using gentamicin without serious side effects is provided.

### SUMMARY OF INVENTION

In a first aspect of the present invention, there is provided a method of attenuating side effects in a patient caused by aminoglycosides, said method including administering aminoguanidine to the patient receiving the aminoglycoside.

The most common side effect of aminoglycosides is nephrotoxicity and renal impairment or kidney damage. The other major side effect associated with the use of aminoglycosides is ototoxicity. The present invention provides a means to attenuate these side effects by administering an aminoguanidine, either before or during treatment with an aminoglycoside. The administration of the aminoguanidine provides a protective effect to prevent the side effects or reduce their effects.

In yet another aspect of the present invention, there is provided a composition when used in the method of attenuating side effects caused by aminoglycosides, said composition comprising a therapeutically effective amount of aminoguanidine to attenuate side effects caused by the aminoglycosides, and a carrier.

In a further preferred embodiment, the composition comprises a combined dosage of aminoguanidine and aminoglycoside for simultaneous administration. The amounts may be such that the aminoglycoside is effective for treating the condition such as an infection by a gram-negative bacteria and the aminoguanidine is effective for attenuating the side effects caused by the aminoglycoside. This combined dosage provides an effective treatment of infection with an effective prevention of side effects during the course of the treatment.

In yet another aspect of the present invention, there is provided a method of treating an infection caused by gram-negative bacteria, said method comprising:

administering an effective amount of an aminoglycoside effective for the treatment of the gram-negative infection; and

administering an effective amount of an aminoguanidine in an amount to attenuate a side effect caused by the aminoglycoside.

In yet another aspect of the present invention, there if provided a kit for the treatment of an infection caused by gram-negative bacterium, said kit comprising:

an amount of aminoglycoside effective for the treatment of the gram-negative bacterium; and

an amount of an aminoguanidine effective to attenuate side effects caused by the aminoglycoside.

### BRIEF DESCRIPTION OF THE FIGURES

FIG. 1 shows the structures of gentamicin and aminoguanidine.

FIG. 2 shows renal mass in experimental models.

FIGS. 3A-C show PAS stain of renal tissue in sham treated animals (3A) control animals treated with gentamicin (3B), control animals treated with gentamicin and aminoguanidine (3C).

FIGS. 4A-C show renal expression of GPX-in sham treated animals (4A) control animals treated with gentamicin (4B), control animals treated with gentamicin and aminoguanidine (4C).

FIG. 5 shows Cortical gentamicin levels after 8-days of treatment # vs gentamicin treated p<0.05).

FIG. 6 shows LMW-AGEs in experimental models following 8-days of treatment # vs gentamicin treated p<0.05).

FIGS. 7A-C show immunostaining for CML-AGE PAS stain of renal tissue from sham treated animals (7A) animals treated with gentamicin (7B), animals treated with gentamicin and aminoguanidine (7C).

FIG. 8 shows MDA-linked fluorescence (top) and AOPPs (bottom) in experimental models following 8-days of treatment (*vs, p<0.05).

### DETAILED DESCRIPTION OF INVENTION

In a first aspect of the present invention, there is provided a method of attenuating side effects in a patient caused by aminoglycosides, said method including administering aminoguanidine to the patient receiving the aminoglycoside.

Aminoglycosides are a group of antibiotics that are used to treat certain bacterial infections. This group of antibiotics includes at least eight drugs: amikacin, gentamicin, kanamycin, neomycin, netilmicin, paromomycin, streptomycin, and tobramycin. All of these drugs have the same basic chemical structure and a similar range of toxic side-effects.

The most common side effect of aminoglycosides is nephrotoxicity and renal impairment or kidney damage. Nephrotoxicity is a term applied to being toxic or destructive to kidney cells. However, nephrotoxicity caused by aminoglycosides may more specifically cause death of tubular cells. It may be distinguished from other causes of kidney damage by distinctive pathology in the mitochondria of tubular cells. Accordingly, to identify specific nephrotoxicities caused by aminoglycosides, in particular gentamicin, its pathology can be compared against other forms of nephrotoxicity. Similarly, the present invention includes within its scope nephrotoxicity of a similar pathology caused by other aminoglycosides that can be attenuated by aminoguanidine. Kidney damage, apparent with changes in urination frequency or urine production, is most likely precipitated by neomycin, tobramycin, and gentamicin. Accordingly, it is preferred that the aminoguanidine attenuates a nephrotoxicity that has a similar pathology to that caused by gentamicin.

The other major side effect associated with the use of aminoglycosides is ototoxicity. Aminoglycosides have been shown to be toxic to certain cells in the ears and particularly in the kidneys. Damage to the middle ear may result in hearing loss or tinnitus. Approximately 5-10% of the people who are treated with aminoglycosides experience some ototoxic side-effect, affecting their hearing or sense of balance. However, in most cases the damage is minor and reversible once medication is stopped. Young children and the elderly are at the greatest risk of suffering side effects. Excessive dosage or poor clearance of the drug from the body can be injurious at any age.

If cells in the inner ear are damaged or destroyed, an individual may also experience a loss of balance and feelings of dizziness. Gentamicin toxicity is the most common single known cause of bilateral vestibulopathy, accounting for 15 to 50% of all cases. Bilateral vestibulopathy, occurs when the balance portions of both inner ears are damaged. The symptoms typically include imbalance and visual symptoms. The imbalance is worse in the dark, or in situations where footing is uncertain. Spinning vertigo is unusual. The visual symptoms, called oscillopsia, only occur when the head is moving. Quick movements of the head are associated with transient visual blurring. This can cause difficulties with seeing signs while driving, or recognizing people's faces while walking. Most people do recover, but the process is slow and usually incomplete. The majority of the improvement occurs at high frequencies with lesser changes seen at lower frequencies. Progression of vestibulotoxicity can occur for months after the last dose, and recovery can be measured out to a year or even longer.

Less common side effects include skin rashes and itching. Very rarely, certain aminoglycosides may cause difficulty in breathing, weakness, or drowsiness. Gentamicin, when injected, may cause leg cramps, skin rash, fever, or seizures. These reactions are generally considered to be idiopathic and not dose-related.

Accordingly, the present invention provides a means to attenuate these side effects by administering an aminoguanidine, either before or during treatment with an aminoglycoside. The administration of the aminoguanidine provides a protective effect to prevent the side effects or reduce their effects.

Preferably the aminoglycosides are selected from the group including amikacin, gentamicin, kanamycin, neomycin, netilmicin, paromomycin, streptomycin, and tobramycin. More preferably the aminoglycoside is gentamicin.

The side effects may be any of the side effects described herein. However, more preferably the side effects of nephrotoxicity or ototoxicity are preferred. Most preferably, aminoguanidine can attenuate the side effects of nephrotoxicity.

Aminoguanidine is known as an inhibitor of Advanced Glycation End (AGE) product formation and inducible Nitric oxide Synthetase (iNOS). The compound may exist in several forms including a hydrochloride, bicarbonate or hemisulphate form. All types of aminoguanidine are useful in the present invention as a similar level of activity has been ascribed to each form.

The aminoguanidine may be administered by the usual routes of administration including orally, rectally, parenterally (that is, intravenously, intramuscularly or subcutaneously), intracisternally, intravaginally, intraperitoneally, topically (as by powders, ointments or drops), transdermally, bucally, or as an oral or nasal spray. Preferably, the method by which the aminoglycoside is administered is the same method by which the aminoguanidine is administered (intravenous, intramuscular or intra-peritoneal) as this may enhance the ability of the aminoguanidine to attenuate any side effects that may result from the aminoglycoside.

Aminoguanidine may be used in any form. However, the aminoguanidine.bicarbonate, aminoguanidine.hydrochloride, or aminoguanidine.hemisulphate may be used. Most preferably the bicarbonate form is used.

An effective dosage of aminoguanidine is a therapeutically acceptable dose, preferably 1 mg/kg/day systemic or preferably 150 mg to 600 mg a day oral/rectal or an equivalent dosage that can deliver the aminoguanidine to a similar concentration in the body. A therapeutically effective dose as used herein refers to that amount of a compound sufficient to result in a healthful benefit in the treated subject. The compound may be the aminoguanidine or the aminoglycoside. However, aminoguanidine delivery by other routes including oral and rectal is also appropriate preferably 150 mg to 600 mg a day. As aminoguanidine has a half life of only 4-hours, so these dosages would be best administered over the day in split doses, preferably the dose is 150 mg to 300 mg a twice daily. Ideally the aminoguanidine is taken with food. Any form of administration may be employed to attenuate the side effects caused by aminoglycosides.

Although these doses and the regimen described may be beneficial, it is contemplated that they be considered as guidelines only and that the attending clinician will determine, in his or her judgement, an appropriate dosage and regimen, based on the patient's age and condition as well as the severity of the condition.

The period of treatment with the aminoguanidine will depend on the treatment of the aminoglycoside. Since the aminoguanidine is used to attenuate any side effects, it may be used before, during or after aminoglycoside treatment. When used before, it is preferably used as a preventative, for preventing or reducing side effects caused by the aminoglycosides. Similarly, the aminoguanidine may be administered during treatment with the aminoglycoside to reduce the side effects or used as a preventative before side effects manifest. If used after the treatment with the aminoglycoside and after the course of the aminoglycoside has been completed, the aminoguanidine may be used to maintain or reduce or attenuate side effects caused by residual aminoglycosides.

When side effects manifest from aminoglycoside treatment, such as that caused by gentamicin treatment, the general course of action is to reduce the dosage or withdraw from the treatment all together. However, this is not ideal if treatment is necessary. Therefore, when aminoguanidine is used in combination with the aminoglycosides, preferably gentamicin, the side effects can be attenuated and treatment continued.

In yet another aspect of the present invention, there is provided a composition when used in the method of attenuating side effects caused by aminoglycosides, said composition comprising a therapeutically effective amount of aminoguanidine to attenuate side effects caused by the aminoglycosides, and a carrier.

The composition comprises aminoguanidine as herein described. Aminoguanidine may be used in any form. However, the aminoguanidine.bicarbonate, aminoguanidine.hydrochloride, or aminoguanidine.hemisulphate may be used. Preferably, the composition comprises bicarbonate of aminoguanidine.

For use as a pharmaceutical, the aminoguanidine and the aminoglycosides are formulated into compositions. The pharmaceutical compositions of this invention can be administered to humans and other animals orally, rectally, parenterally (i.e. intravenously, intramuscularly, or sub-cutaneously), intracisternally, intravaginally, intraperitoneally, topically (as by powders, ointments, or drops), transdermally, bucally, or as an oral or nasal spray.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If desired, and for more effective distribution, the compounds can be incorporated into slow release or targeted delivery systems such as polymer matrices, liposomes, and microspheres. This may be particularly useful for the slower administration of the aminoguanidine as a preventative against side effects caused by the aminoglycosides.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

If desired, and for more effective distribution, the compounds can be incorporated into slow release or targeted delivery systems such as polymer matrices, liposomes, and microspheres.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, sprays, ointments and inhalants. The active compound is mixed under sterile conditions with a pharmaceutically acceptable carrier and any needed preservatives, buffers, or propellants which may be required.

The dosage of the aminoguanidine will depend on the usage. For instance, if the aminoguanidine is to be administered along with the aminoglycoside treatment, unit dosage amounts sufficient to attenuate the side effects can be included in the composition to ensure sufficient aminoguanidine in the presence of the aminoglycoside. Given the half-life of the aminoguanidine, it is preferable that the composition include unit dosage forms for regular administration.

Preferably, the composition ensures that there is an effective dose of approximately 150 mg to 600 mg per day. Preferably, the composition comprises a dosage of 150 mg to 300 mg twice daily.

In a further preferred embodiment, the composition comprises a combined dosage of aminoguanidine and aminoglycoside for simultaneous administration. The amounts may be such that the aminoglycoside is effective for treating the condition such as an infection by a gram-negative bacteria and the aminoguanidine is effective for attenuating the side effects caused by the aminoglycoside. This combined dosage provides an effective treatment of infection with an effective prevention of side effects during the course of the treatment.

In yet another aspect of the present invention, there is provided a method of treating an infection caused by gram-negative bacteria, said method comprising:

administering an effective amount of an aminoglycoside effective for the treatment of the gram-negative infection; and

administering an effective amount of an aminoguanidine in an amount to attenuate a side effect caused by the aminoglycoside.

This aspect is particularly effective when a treatment is required over a prolonged period. Over extended periods, aminoglycosides such as gentamicin will cause side effects. This combined treatment of aminoguanidine with aminoglycosides will allow for extended treatments without or with reduced side effects. The period over which the method can be applied may be for as long as the aminoglycoside treatment.

The aminoguanidine and the aminoglycosides can be administered alone or in combination. When administered alone, the aminoglycosides and the aminoguanidine may be administered in unit dosage forms or in slow release forms to ensure a continued administration of the two compounds for effective and prolonged treatment of the infection caused by the gram-negative bacterium.

Concentrations of the aminoguanidine are as herein described. Methods of administering the compounds as well as the compositions used in the treatments are as herein described.

In yet another aspect of the present invention, there if provided a kit for the treatment of an infection caused by gram-negative bacterium, said kit comprising:

an amount of aminoglycoside effective for the treatment of the gram-negative bacterium; and

an amount of an aminoguanidine effective to attenuate side effects caused by the aminoglycoside.

The aminoglycoside and the aminoguanidines can be used separately or in combination. The methods of administration of the two compounds may be as herein described. The aminoguanidine may be administered to a patient before, during or after the aminoglycoside treatment.

In a further preferred form of the kit, there is included a set of instructions for the use of the aminoglycoside and the aminoguanidine for the treatment of the gram-negative bacterial infection. Methods of administration are as herein described including the amounts of aminoguanidine useful for the attenuation of side effects caused by the aminoglycosides.

Throughout the description and claims of this specification the word “comprise”, and variations of the word such as “comprising” and “comprises”, is not intended to exclude other additives or components or integers or steps.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

Examples of the procedures used in the present invention will now be more fully described. It should be understood, however, that the following description is illustrative only and should not be taken in any way as a restriction on the generality of the invention described above.

### EXAMPLES

### Example 1

### Effect of Aminoguanidine on Gentamycin Toxicity

This example documents that the AGE-inhibitor, aminoguanidine, was protective against tubular toxicity when co-administered with gentamicin.

1. Materials and Methods

(a) Animal Treatments and Specimen Collection

Male Sprague-Dawley rats were randomly assigned to one of three groups:

Group 1 (Gentamicin): These animals received timed daily subcutaneous injections of gentamicin (50 mg/kg/d) in divided doses, delivered during the resting period, for 8 consecutive days.

Group 2 (Gentamicin+aminoguanidine): rats received aminoguanidine 4 g/L in their drinking water for 2 weeks, prior to gentamicin dosing as described above.

Group 3 (Sham): These animals received twice daily subcutaneous injections of equivalent amount of vehicle (0.9% NaCl) according to the same timed protocol for 8 consecutive days.

Prior to gentamicin dosing and on day 7 of the study, animals were placed individually in metabolic cages and 24 h urine collected and food intake recorded. Blood was collected into heparinised tubes, after being obtained from the tail of each rat, at baseline and on days 2 and 5. The blood samples were always obtained before the daily injections were given, as well as being taken 24 hours following the last injection and at the time of sacrifice. At the end of day 8 (i.e., morning of day 9), and twenty-four hours after the last gentamicin (or normal saline) injection, rats were killed by decapitation. Kidneys were harvested and blood collections were performed. Serum was separated from these blood samples and used for the measurements described below. One kidney was immediately removed for histological examination.

(b) Parameters Measured

Standard physiological and biochemical parameters including body weight and food and water intake, were measured before and after gentamicin dosing regimens. Serum and urine samples obtained at the same time points were tested for standard biochemical indices (Table 1.1), using a Hitachi 747 Automatic Analyser. (Roche Diagnostics, Basel, Switzerland). Serum gentamicin was measured in serum by a polarisation fluorescence immunoassay in a TDx analyser (Abbott Diagnostics, Abbott Park, Ill., USA). Gentamicin standards were prepared in normal rat sera. As noted above, tubular enzymuria has been used as a valid marker of gentamicin toxicity. To quantify enzymuria, urinary excretion of alanine transaminase (ALT), aspartate transferase (AST), alkaline phosphatase (AP) and lactate dehydrogenase (LDH) were measured using a Hitachi 747 Automatic Analyser, as above. Various tubular syndromes with electrolyte wasting have been described after long term gentamicin treatment in older children and adults. A recent study in adults demonstrated a fourfold increase of calcium (Ca) and magnesium (Mg) excretion after a single dose of gentamicin, before any other evidence of toxicity was observed. Therefore the urinary excretion of the electrolytes were measured in this experimental protocol and expressed as the fractional excretion (FE), which is calculated as:
FEₓ(%)=(Uₓ×P_{Cr})/(Pₓ×U_{Cr})

where Ux and Px are the concentrations of the substance in the urine and plasma, respectively, and UCr and PCr are the concentrations of creatinine in urine and plasma, respectively. Serum and urine creatinine were measured by HPLC. Creatinine clearance was calculated by the standard equation and expressed in ml/min. As noted above, renal impairment in gentamicin toxicity results from a reduction in effective renal plasma flow, possibly due to activation of the intra-renal RAS. As an additional approach to measure this effect, ERPF was estimated from the clearance of endogenous hippuric acid clearance in conscious animals.

TABLE 1.1
Serum and urine parameters measured in the experiment
Serum parameters

| | |
|---|---|
| Sodium (Na) | Glycated Hb (GHb) |
| Potassium (K) | Alanine transaminase (ALT), |
| Magnesium (Mg) | Aspartate transaminase (AST), |
| Calcium (Ca) | Alkaline phosphatase (AP) |
| Albumin | Lactate dehydrogenase (LDH) |
| Urea nitrogen | Creatinine clearance |
| Creatinine | Glucose |

Urine parameters

| | |
|---|---|
| Fractional potassium excretion | Enzymuria: |
| Fractional magnesium excretion | Alanine transaminase (ALT), |
| Fractional calcium excretion | Aspartate transaminase (AST), |
| Total proteinuria | Alkaline phosphatase (AP) |
| Albuminuria | Lactate dehydrogenase (LDH) |

As a marker of renal NO generation, daily excretion of urinary nitrate/nitrite (NOₓ) was measured using the Griess reaction (Komers et al., 1994; Miranda et al., 2001). Briefly, 0.5 ml of urine was centrifuged at 10,000 g for 15 minutes, the supernatant removed and diluted fivefold using deionised water. From each sample 50 μl was dispensed into a 96-well plate followed by 50 μl of freshly prepared vanadium chloride solution (0.8 mg/ml in 1M HCl) and immediately followed by 50 μl of Griess reagent (2% sulphanilamide in 1.47M hydrochloric acid) and 0.1% N-(1-aphthyl)ethylenediamine dihydrochloride in deionised water. After 30 minutes of colour development at 37° C., absorbance was measured on a microplate spectrophotometer (Emax, Molecular Biosystems, Mo., USA) at 570 nm with a 630 nm reference filter. Each sample was assayed in triplicate. Urinary NOx excretion was expressed as μmol/mg creatinine.

Recent studies have also suggested that serum ACE activity is a reliable marker of gentamicin toxicity (Ziai et al., 2003). Consequently, serum ACE activity was estimated in animals before and after administration of gentamicin using the synthetic ACE-specific substrate, hippuryl-histidyl-leucine (Shihabi and Scaro, 1981). The released product (His-Leu) was fluorimetrically measured after reaction with ophthaldehyde in time-fixed assays. Briefly, 20 μl of 10 times diluted plasma was mixed with 20 μl Hip-His-Leu (final conc.=1 mM) made up to 50 μl with 10 μL of 100 mM Tris-HCl, 300 mM NaCl, pH 8.3. The samples were then mixed and incubated in 37° C. water bath for 30 minutes. To detect the generated His-Leu, 120 μl of 0.3 N NaOH and 10 μl o-phthaldehyde were then added and the samples briefly vortexed and incubated at room temperature for 10 minutes. To terminate the derivitisation reaction, 20 μl of 3N HCl was added and the samples centrifuged for 5 minutes at 15, 000 rpm to remove insoluble residue. The resulting supernatant was transferred to a black 96 well plate and fluorescence read at excitation=355 nm, emission=485 nm in a fmax fluorescence detector (Molecular Biosystems, USA). Results were corrected for blank fluorescence detected in the absence of Hip-His-Leu. Results are expressed as nmol of His-Leu generated per minute of reaction time per ml of plasma (nmol/min/ml).

(c) Morphological Examination of the Kidney

Paraffin embedded sections were stained with periodic acid-Schiff (PAS) and examined by light microscopy to determine the extent of gentamicin toxicity (Kopple et al., 2002). In addition, tubular apoptosis was assessed by staining for DNA fragmentation using propidium iodide. Briefly, sections (3 μm) from the same paraffin blocks used for PAS staining were de-paraffinised with xylene and rehydrated, soaked with PBS pH 7.4 and incubated for 10 minutes with propidium iodide 10 μg/ml and RNAse A 100 μg/ml at 37° C. Sections were subsequently washed extensively with PBS, rinsed with distilled water and mounted with an aqueous media (AFT, Behring Diagnostics, Somerville, N.J.). On a fluorescence microscope, renal tissue was identified on bright field and then, using a rhodamine filter set, the normal and apoptotic tubular nuclei were counted. Twenty cortical windows were assessed in each animal. The results were expressed as a mean number of fragmented and condensed nuclei (apoptotic cells) per window. As a marker of proximal tubular injury, renal gene expression of extracellular glutathione peroxidase, GPX-3 (Yazar et al., 2003) was also assessed in paraffin fixed sections by in situ hybridization

(d) Determination of Tissue Gentamicin Level

Gentamicin content was also measured in the cortex of the kidney because the majority of gentamicin is reported to accumulate in the renal cortex, primarily in the proximal tubule. To measure the gentamicin concentrations in the renal cortex, the kidney was processed and analysed. The upper pole of the vertically divided right kidney was removed and snap-frozen at minus 80° C. until use. The cortical tissue was homogenised in 3 ml of 0.15M trichloroacetic acid at 4° C. After centrifugation, a 100 μl aliquot of the supernatant was used immediately for gentamicin determination by HPLC. Briefly, gentamicin in samples was derivitised with o-phthalaldehyde and 3-mercaptopropionic acid and then injected into a mobile phase consisting of methanol-glacial acetic acid-water (800:20:180, v/v) containing 0.02M sodium heptanesulfonic acid, pH 3.4, flowing at 1.0 ml/min. Separation was performed on a μBondopak, C18 column (300×3.9 mm i.d., particle size 10 μm, Waters, USA) with a C18 pre-column. Fluorescence detection was set at excitation 340 nm, emission 418 nm. The C1a gentamicin peak was used to quantify the total gentamicin content. Calibration curves of the components were prepared in a drugfree supernatant of the renal homogenates and were linear over the concentration range 50-500 μg/ml. The day-to-day reproducibility was <6%. Renal cortical gentamicin concentrations were expressed per milligram of renal cortical protein.

(e) Oxidation, Lipoxidation and Glycation Products

Serum and tissue advanced oxidation products (AOPP) were measured by spectrophotometric methods (Martin-Gallan et al., 2003). This assay largely detects the presence of protein dityrosine modifications (Witko-Sarsat et al., 2003). Briefly, 200 μl of homogenised renal tissue or serum was diluted 1:3 in PBS, 100 μL of 1.16 mol/L potassium iodide (KI, Sigma) were then added to each tube, followed two minutes later by 200 μL of concentrated acetic acid. The absorbance of the reaction mixture was immediately read at 340 nm against a blank containing 200 μL of PBS, 100 μL of KI, and 200 μL of acetic acid. Concentrations of AOPP were expressed as arbitrary units calibrated to a chloramine T standard.

Tissue-linked glycoxidation products were measured, based on tissue fluorescence using a flow injection assay (Wrobel et al., 1998). Briefly, renal tissue was enymatically hydrolysed using proteinase K. The solution was then centrifuged to remove sediment and the aqueous phase injected directly into a water column flowing through a fluorescence detector (Waters 470, Waters USA) and UV detector (Hewlett-Packard, USA) in series. Pentosidine-linked fluorescence was measured at excitation 325 nm with an emission at 385 nm (Meng et al., 2000). The level of lipid peroxidation was estimated by determining the malondialdehyde (MDA)-linked fluorescence (390 nm/emission 460 nm) (Meng et al., 1998). The values of fluorescence were corrected against those of the blanks containing the same concentration of proteinase K and adjusted for the peptide content using the absorbance at 280 nm. The data were expressed in arbitrary units, with samples being given the reference value of 1. Finally, paraffin sections were stained for CML-AGEs

(f) Bactericidal Studies

To confirm that aminoguanidine did not interfere with the antibiotic efficacy of gentamicin, the minimum inhibitory concentration (MIC) was determined for Escherichia coli (ATCC 25922) in the presence and absence of aminoguanidine. Briefly, to 20 mls of Mueller-Hinton broth, supplemented with calcium and magnesium, aminoguanidine was added at three different final concentrations (0.1, 1, 10 μg/ml), consistent with the possible range achieved in human serum (Tilton et al., 1993). A standardised E coli (ATCC 25922) was then inoculated into the broth and the lowest concentration of gentamicin to inhibit the growth of the bacterium determined (MIC). Results are expressed in milligrams of gentamicin per litre. Studies were performed in duplicate on two separate occasions by a blinded operator. A clinically significant change was defined as an increase in the MIC by two or more doubling dilutions.

2. Results

(a) Baseline Functional Parameters

Pre-treatment with aminoguanidine did not significantly modify the majority of the serum or urine parameters in animals prior to the commencement of gentamicin therapy (Table 1.2 and Table 1.3). In addition, baseline GFR was not significantly different among the treatment groups. However, serum magnesium levels were significantly lower in animals treated with aminoguanidine (p<0.05) Urinary potassium excretion was reduced by aminoguanidine, as was the urinary excretion of AST (both p<0.05).

TABLE 1.2
Serum parameters after aminoguanidine
pre-treatment but before gentamicin therapy

| | | | Control + |
|---|---|---|---|
| | Control | Control + gentamicin | AG + gentamicin |
| Sodium | 144 ± 1 | 145 ± 1 | 143 ± 1 |
| (mmol/L) | | | |
| Potassium | 4.9 ± 0.1 | 4.7 ± 0.1 | 4.9 ± 0.2 |
| (mmol/L) | | | |
| Magnesium | 0.93 ± 0.05 | 0.88 ± 0.03 | 0.76 ± 0.01# |
| (mmol/L) | | | |
| Creatinine | 32 ± 1 | 32 ± 1 | 31 ± 1 |
| (μmol/L) | | | |
| Urea | 7.0 ± 0.2 | 7.0 ± 0.2 | 7.2 ± 0.4 |
| (mmol/L) | | | |
| Protein | 72 ± 2 | 74 ± 1 | 72 ± 1 |
| (mg/ml) | | | |
| Glucose | 6.9 ± 0.3 | 6.3 ± 0.2 | 7.1 ± 0.3 |
| (mmol/L) | | | |
| Calcium | 2.71 ± 0.04 | 2.73 ± 0.02 | 2.72 ± 0.02 |
| (mmol/L) | | | |
| LDH | 167 ± 20 | 188 ± 10 | 137 ± 12 |
| (IU/ml) | | | |
| ALT | 56 ± 3 | 58 ± 5 | 48 ± 4 |
| (IU/ml) | | | |
| AST | 85 ± 3 | 89 ± 7 | 81 ± 4 |
| (IU/ml) | | | |
| AP | 237 ± 21 | 222 ± 23 | 246 ± 26 |
| (IU/ml) | | | |
| HbA_{1c} | 3.8 ± 0.1 | 3.6 ± 0.1 | 3.9 ± 0.1 |
| (%) | | | |

TABLE 1.3
Urine parameters after aminoguanidine
pre-treatment but before gentamicin therapy

| | | | Control + |
|---|---|---|---|
| | Control | Control + gentamicin | AG + gentamicin |
| Urine volume | 17 ± 2 | 18 ± 2 | 21 ± 2 |
| (ml/day) | | | |
| FE Mg | 5 ± 1 | 5 ± 1 | 6 ± 2 |
| (%) | | | |
| FE K | 16 ± 1 | 16 ± 1 | 16 ± 1 |
| (%) | | | |
| FE Ca | 0.3 ± 0.1 | 0.3 ± 0.1 | 0.5 ± 0.1 |
| (%) | | | |
| Protein | 14 ± 1 | 18 ± 2 | 15 ± 1 |
| (μg/min) | | | |
| LDH | 227 ± 35 | 290 ± 45 | 263 ± 32 |
| (mU/min) | | | |
| ALT | 12 ± 1 | 20 ± 1 | 16 ± 2 |
| (mU/min) | | | |
| AST | 120 ± 18 | 131 ± 12 | 76 ± 11# |
| (mU/min) | | | |
| ALP | 640 ± 25 | 803 ± 68 | 933 ± 86 |
| (mU/min) | | | |

(b) Enzymuria Following Gentamicin Treatment

Consistent with the induction of nephrotoxicity, rats treated with gentamicin alone developed significant enzymuria with increased urinary excretion of LDH, AST, ALT and alkaline phosphatase (Table 1.4). Rats treated with both aminoguanidine and gentamicin had levels of enzymuria not significantly different from animals with the exception of the urinary excretion of LDH, which, though significantly reduced in comparison to animals that received gentamicin alone, did not return to sham values. In addition, an increase in proteinuria occurred only in the animals treated with gentamicin alone, representing the increased excretion of tubular proteins but not albumin. Urine volumes increased from baseline levels in gentamicin-treated animals (p<0.01).

TABLE 1.4
Urine parameters in experimental models following 8-days of treatment

| Urine | Volume | LDH | ALT | AST | AP | Albumin | Protein |
|---|---|---|---|---|---|---|---|
| parameters | (ml/day) | (U/ml) | (U/ml) | (U/ml) | (U/ml) | (μg/day) | (μg/day) |
| Sham | 18 ± 2 | 18 ± 2 | 1.5 ± 0.2 | 82 ± 3 | 66 ± 17 | 0.3 ± 0.1 | 1.1 ± 3 |
| Gentamicin | 29 ± 6* | 489 ± 100* | 10.5 ± 4.6* | 147 ± 62* | 119 ± 29* | 0.4 ± 0.1 | 2.5 ± 4* |
| Gentamicin + AG | 30 ± 3* | 95 ± 17*# | 2.2 ± 0.5 | 91 ± 5 | 61 ± 28 | 0.3 ± 0.1 | 1.2 ± 2 |

(Mean ± SEM; *vs sham p < 0.05; #vs gentamicin treated p < 0.05)

(c) Markers of Renal Function

Following treatment with gentamicin alone, animals developed significant renal impairment, with a fall in creatinine clearance to greater than 50% when compared to untreated animals (Table 1.5). This was associated with a significant reduction in renal blood flow as estimated by the clearance on endogenous hippurate (8.1±1.1 ml/min) compared to untreated animals (12.6±1.6 ml/min, p<0.05). Treatment with aminoguanidine attenuated these changes. Notably creatinine clearance was restored to sham levels and renal blood flow increased to the same extent as seen in untreated shams (12.1±1.1 ml/min). In addition, the fractional excretion of potassium and magnesium was significantly elevated in animals treated with gentamicin alone, as the result of gentamicin-induced tubular dysfunction (Table 1.5). Treatment with aminoguanidine again attenuated these changes, although it did not return these indices to baseline levels.

TABLE 1.5
Renal function markers in experimental
models following 8-days of treatment

| | Creatinine | Urea | Creatinine | FeK | FeCa | FeMg |
|---|---|---|---|---|---|---|
| Control | (μM) | (mM) | clearance | (%) | (%) | (%) |
| Sham | 33 ± 2 | 7.5 ± 0.4 | 3.3 ± 0.3 | 20 ± 2 | 1 ± 1 | 5 ± 1 |
| Gentamicin | 122 ± 55* | 20.1 ± 5.6* | 1.3 ± 0.3 | 70 ± 20* | 6 ± 3 | 21 ± 5* |
| Gentamicin + AG | 40 ± 3 | 6.7 ± 0.3 | 3.0 ± 0.2 | 32 ± 5*# | 2 ± 1* | 10 ± 1*# |

(Mean ± SEM; *vs sham p < 0.05; #vs gentamicin treated p < 0.05)

(d) Other Markers of Renal Injury

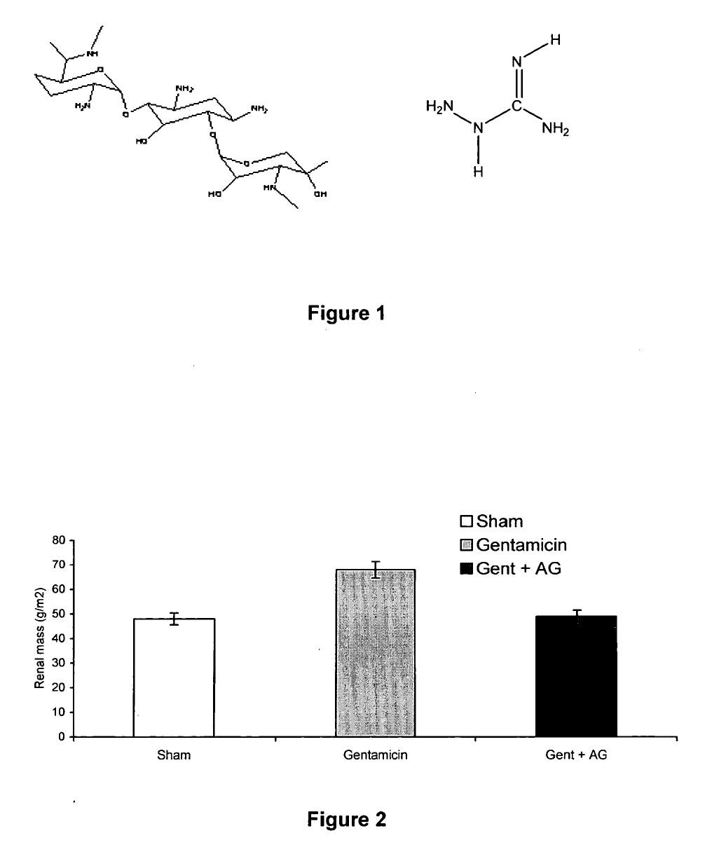

Animals treated with gentamicin alone had significantly higher normalised kidney weights than untreated animals (FIG. 2). Treatment with aminoguanidine reduced the renal weight, consistent with the prevention of renal oedema associated with gentamicin toxicity.

Serum markers of tissue injury were also elevated in animals treated with gentamicin (Table 1.6). Both serum LSH and AST were elevated in animals treated with gentamicin. Again, this was prevented by treatment with aminoguanidine. In addition, serum ACE activity was significantly lower in animals receiving gentamicin alone, when compared with the sham treated animals. Treatment with aminoguanidine also prevented this decline.

TABLE 1.6
Markers of renal injury in experimental models
(* vs sham, p < 0.05; # vs gentamicin treated, p < 0.05)

| | LDH | AST | ACE activity | Urine NOx |
|---|---|---|---|---|
| Control | (IU/ml) | (IU/ml) | nmol/ml/min, | μmol/mg |
| Sham | 185 ± 10 | 84 | 7.4 ± 0.5 | 0.4 ± 0.1 |
| Gentamicin | 380 ± 76* | 216* | 5.1 ± 0.4* | 4.1 ± 0.4* |
| Gentamicin + AG | 186 ± 14 | 84 | 7.2 ± 0.4 | 0.6 ± 0.2 |

In addition, urinary NOx excretion was significantly increased in animals treated with gentamicin alone, consistent with the induction of renal iNOS associated with gentamicin toxicity (Rivas-Cabanero et al., 1997). Aminoguanidine, an inhibitor of iNOS, normalised the urinary excretion of NOx in animals.

(e) Histological Evidence of Renal Injury

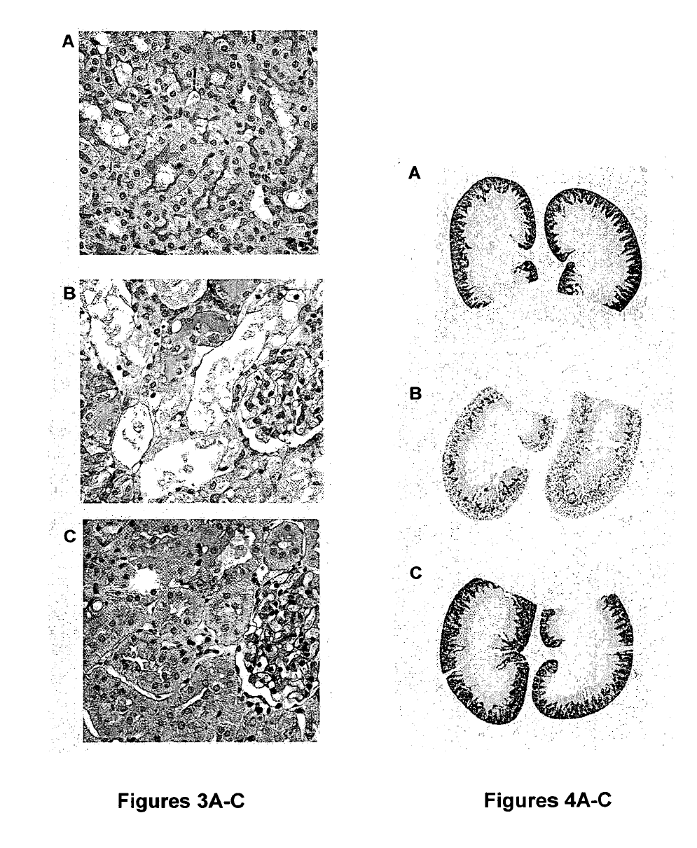

Rats receiving gentamicin alone developed significant renal injury, with histological evidence of extensive and marked tubular necrosis throughout the cortex (FIG. 3). In addition, severe necrosis (manifested by complete absence of the tubular profile, white arrow) was only present in animals treated with gentamicin alone. Mild cortical changes were apparent in animals treated with aminoguanidine and gentamicin, although tubular necrosis was absent from this group. Correlating with histological changes of gentamicin toxicity, tubular nuclei in animals treated with gentamicin contained 12±2 fragmented and condensed nuclei (apoptotic cells) per field. This compared to less than 1 apoptotic nuclei per field in sham treated animals and 1-2 nuclei per field in animals receiving aminoguanidine. Finally, the tubular expression of glutathione peroxidase-3, a known marker of proximal tubular synthetic function was significantly reduced in animals treated with gentamicin alone, but not significantly changed in animals that received aminoguanidine (FIG. 4).

(f) Serum and Renal Gentamicin Levels

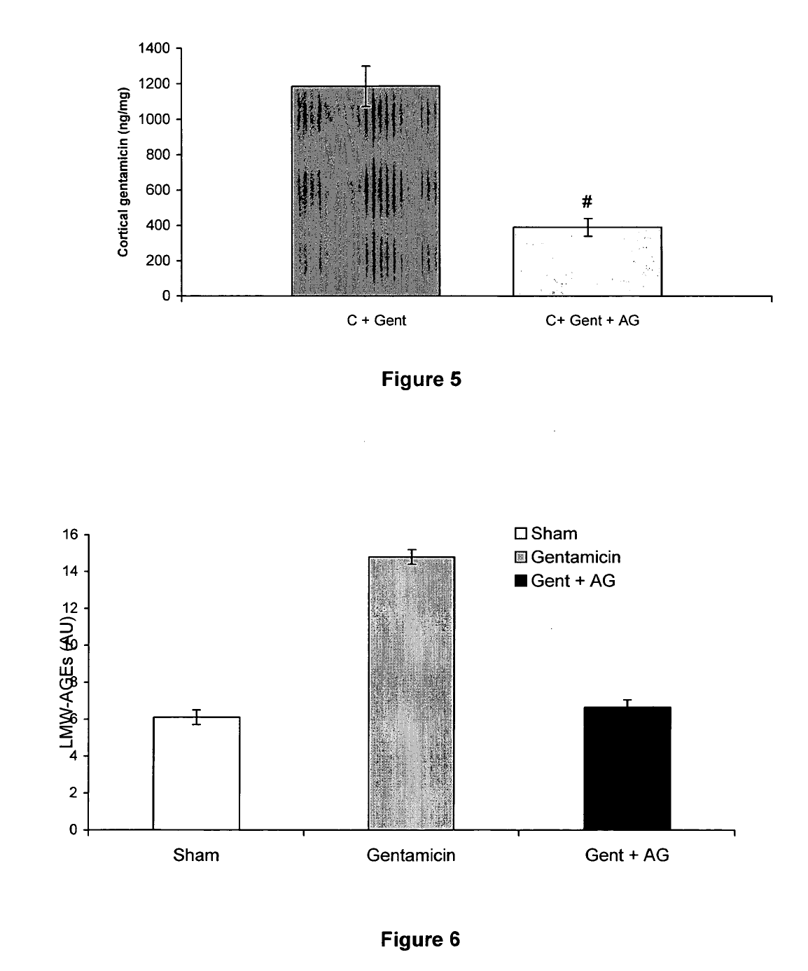

The gentamicin content of the renal cortex was elevated in animals treated with gentamicin, consistent with studies showing the uptake-dependent of renal injury in gentamicin toxicity (FIG. 5). Treatment with aminoguanidine in animals reduced gentamicin content by approximately two thirds (p<0.001). At the same time, serum levels at day 2 and day 5 were unaffected by co-administration of aminoguanidine. This implies a real reduction in gentamicin uptake induced by aminoguanidine. 20 The cortical gentamicin concentration was strongly correlated with parameters of renal damage. For example, cortical gentamicin content was negatively correlated with GFR(R2=0.72, p<0.01) and plasma AST (R2=0.49, p<0.01) in animals treated with gentamicin.

(g) Advanced Glycation End Products

Although gentamicin had no effect on glycaemic, circulating levels of LMW-AGEs in animals, following the induction of gentamicin toxicity, were elevated to over twice the level seen in untreated animals. Again treatment with aminoguanidine attenuated this effect in animals.

Staining for N-carboxymethylysine (CML) was substantially increased in animals treated with gentamicin (FIG. 6), reflecting the marked increase in oxidative and, in particular, lipoxidative stress associated with aminoglycoside toxicity. Notably, there was no accumulation of AGE-fluorescence in these damaged kidneys.

These results show that aminoguanidne was also able to reduce staining for CML-AGE in kidneys treated with gentamicin. While this may largely represent the reduced uptake of gentamicin, an additional and beneficial protection, afforded by aminoguanidine, related to this antioxidant effect, as well as its ability to scavenge peroxynitrite radicals. Aminoguanidine was also able to prevent the accumulation of fluorescent LMWAGEs in the plasma of animals treated with gentamicin. This accumulation most likely represents the reduction in renal clearance of these moieties that may be associated with nephrotoxicity. Furthermore, a reduction in LMW-AGEs is linked to renoprotection in this model. However, an important feature of this finding is that the accumulation of plasma LMW-AGEs was clearly dissociated from the level of tissue AGE-fluorescence. Although generally regarded as a marker of tissue AGE-burden, this finding implies a much more dynamic regulation of LMW-AGE levels. It also has implications for the interpretation of LMW-AGEs measured in the clinical setting.

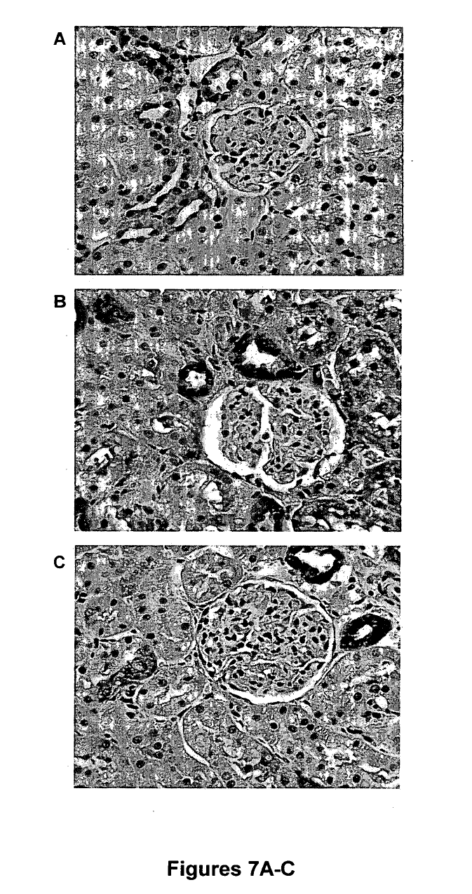

Cortical AGE-fluorescence, following the induction of gentamicin toxicity in animals, was not significantly different from untreated animals (1.0±0.3; C+gent 1.2±0.3, p=NS). In addition, tissue AGE fluorescence failed to correlate with circulating LMW-AGEs in gentamicin treated animals (R=0.0, p=0.8). Against this, tubular immunostaining for CML-AGE was substantially increased in animals treated with gentamicin (FIG. 7).

h) Oxidative Modification

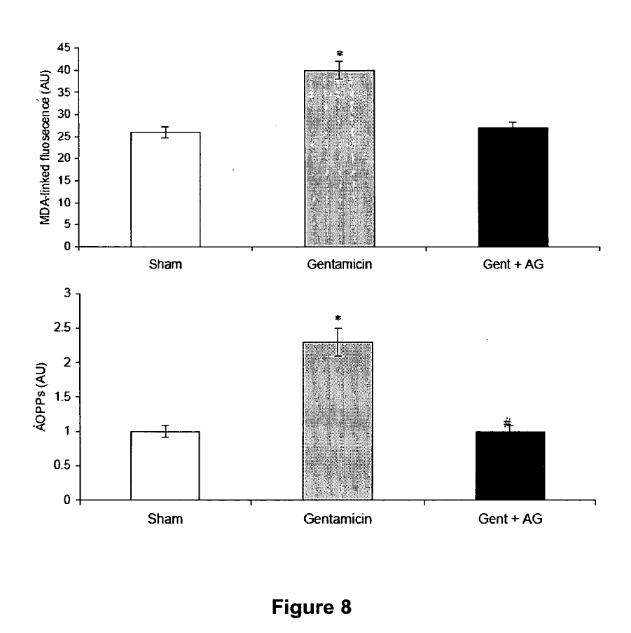

The induction of oxidative stress in animals treated with gentamicin was determined by measuring both serum MDA-linked fluorescence and advanced oxidation products (AOPPs). As predicted, MDA-linked fluorescence was elevated in animals treated with gentamicin alone compared to sham animals or those receiving aminoguanidine (FIG. 8). Serum AOPPs, which predominantly measure dityrosine modifications, were elevated to approximately the same extent (FIG. 8). Both these changes were correlated with changes in circulating LMW-AGEs (vs MDA-linked fluorescence, R²=0.75, p<0.001; vs AOPPs R²=0.68, p<0.001) and with each other (R²=0.75).

(i) Antibiotic Efficacy

Supplementation of aminoguanidine to gentamicin did not significantly interfere with the antibiotic efficacy of this agent. The MIC for gentamicin in the absence of aminoguanidine (0.2 μg/ml) was not significantly modified in the presence of aminoguanidine at 1 mg/ml (MIC, 0.5 μg/ml), 100 μg/ml (MIC, 0.2 μg/ml) or 10 μg/ml (MIC, 0.3 μg/ml). Although the MIC was slightly greater with the high dose of aminoguanidine, this MIC is within the normal range for the organisms and a change of this magnitude is of no clinical significance.

### REFERENCES

Each of the following references is hereby expressly incorporated by reference in its entirety.
Komers R, Allen T J, Cooper M E. Role of endothelium-derived nitric oxide in the pathogenesis of the renal hemodynamic changes of experimental diabetes. Diabetes. 1994; 43(10): 1190-7.
Kopple J D, Ding H, Letoha A, Ivanyi B, Qing D P, Dux L, Wang H Y, Sonkodi S. L-carnitine ameliorates gentamicin-induced renal injury in rats. Nephroi Dial Transplant. 2002; 17(12): 2122-31.
Martin-Gallan P, Carrascosa A, Gussinye M, Dominguez C. Biomarkers of diabetes-associated oxidative stress and antioxidant status in young diabetic patients with or without subclinical complications. Free Radic Biol Med. 2003; 34(12): 1563-74.
Meng J, Sakata N, Takebayashi S, Asano T, Futata T, Nagai R, Ikeda K, Horiuchi S, Myint T, Taniguchi N. Glycoxidation in aortic collagen from STZ-induced diabetic rats and its relevance to vascular damage. Atherosclerosis. 1998; 136(2): 355-65.
Miranda K M, Espey M G, Wink D A. A rapid, simple spectrophotometric method for simultaneous detection of nitrate and nitrite. Nitric Oxide. 2001; 5(1): 62-71.
Shihabi Z K, Scaro J. Liquid-chromatographic assay of angiotensin-converting enzyme in serum. Clin Chem. 1981 October; 27(10):1669-71
Tilton R G, Chang K, Hasan K S, Smith S R, Petrash J M, Misko T P, Moore W M, Currie M G, Corbett J A, McDaniel ML. Prevention of diabetic vascular dysfunction by guanidines. Inhibition of nitric oxide synthase versus advanced glycation end-product formation. Diabetes. 1993; 42(2): 221-32.
Witko-Sarsat V, Gausson V, Descamps-Latscha B. Are advanced oxidation protein products potential uremic toxins? Kidney Int Suppl. 2003; (84): S11-4.
Wrobel K, Wrobel K, Garay-Sevilla M E, Nava L E, Malacara J M. Novel analytical approach to monitoring advanced glycosylation end products in human serum with on-line spectrophotometric and spectrofluorometric detection in a flow system. Clin Chem. 1997; 43(9): 1563-9.
Yazar E, Elmas M, Altunok V, Sivrikaya A, Oztekin E, Birdane Y O. Effects of aminoglycoside antibiotics on renal antioxidants, malondialdehyde levels, and some serum biochemical parameters. Can J Vet Res. 2003; 67(3): 239-40.
Ziai S A, Salehian P, Mahmoudian M. Study of serum and tissues angiotensin converting enzyme (ACE) activity in rat with gentamicin induced renal toxicity. Ren Fail. 2003; 25(6): 923-33.

Finally it is to be understood that various other modifications and/or alterations may be made without departing from the spirit of the present invention as outlined herein.

1. A method of attenuating side effects in a patient caused by aminoglycosides, said method including administering aminoguanidine to the patient receiving the aminoglycoside.

2. A method according to claim 1 wherein the side effect is nephrotoxicity or ototoxicity.

3. A method according to claim 1 wherein the side effect is nephrotoxicity.

4. A method according to claim 1 wherein the aminoguanidine is administered before or during treatment with the aminoglycoside.

5. A method according to claim 1 wherein the aminoguanidine is administered at 1 mg/kg/day.

6. A method according to claim 1 wherein the aminoguanidine is administered at 150 mg to 600 mg/day.

7. A method according to claim 1 wherein the aminoguanidine is aminoguanidine bicarbonate, aminoguanidine hydrochloride or aminoguanidine hemisulphate.

8. A method according to claim 1 wherein the aminoguanidine is aminoguanidine bicarbonate.

9. A method according to claim 1 wherein the aminoglycoside is selected from the group including amikacin, gentamicin, kanamycin, neomycin, netilmicin, paromomycin, streptomycin, and tobramycin.

10. A method according to claim 1 wherein the aminoglycoside is gentamicin.

11. A composition for use in attenuating side effects caused by aminoglycosides, said composition comprising a therapeutically effective amount of aminoguanidine to attenuate side effects caused by the aminoglycosides, and a carrier.

12. A composition according to claim 11 wherein the side effect is nephrotoxicity or ototoxicity.

13. A composition according to claim 11 wherein the side effect is nephrotoxicity.

14. A composition according to claim 11 comprising a combined dosage of aminoguanidine and aminoglycoside for simultaneous administration.

15. A composition according to claim 11 wherein the aminoguanidine is aminoguanidine bicarbonate, aminoguanidine hydrochloride, or aminoguanidine hemisulphate.

16. A composition according to claim 11 wherein the aminoguanidine is aminoguanidine bicarbonate.

17. A composition according to claim 11 wherein the aminoglycoside is selected from the group including amikacin, gentamicin, kanamycin, neomycin, netilmicin, paromomycin, streptomycin, and tobramycin.

18. A composition according to claim 11 wherein the aminoglycoside is gentamicin.

19. A composition according to claim 14 wherein the aminoglycoside is selected from the group including amikacin, gentamicin, kanamycin, neomycin, netilmicin, paromomycin, streptomycin, and tobramycin.

20. A composition according to claim 14 wherein the aminoglycoside is gentamicin.

21. A method of treating an infection caused by a gram-negative bacteria, said method comprising:
administering an effective amount of an aminoglycoside effective for the treatment of the gram-negative infection; and
administering an effective amount of an aminoguanidine in an amount to attenuate a side effect caused by the aminoglycoside.

22. A method according to claim 21 wherein the side effect is nephrotoxicity or ototoxicity.

23. A method according to claim 21 wherein the side effect is nephrotoxicity.

24. A method according to claim 21 wherein the administering of the aminoglycoside and aminoguanidine is a combined treatment administered simultaneously.

25. A method according to claim 21 wherein the aminoguanidine is administered before or during treatment with the aminoglycoside.

26. A method according to claim 21 wherein the aminoguanidine is administered at 1 mg/kg/day.

27. A method according to claim 21 wherein the aminoguanidine is administered at 150 mg to 600 mg/day.

28. A method according to claim 21 wherein the aminoguanidine is aminoguanidine bicarbonate, aminoguanidine hydrochloride or aminoguanidine hemisulphate.

29. A method according to claim 21 wherein the aminoguanidine is aminoguanidine bicarbonate.

30. A method according to claim 21 wherein the aminoglycoside is selected from the group including amikacin, gentamicin, kanamycin, neomycin, netilmicin, paromomycin, streptomycin, and tobramycin.

31. A method according to claim 21 wherein the aminoglycoside is gentamicin.

32. A kit for the treatment of an infection caused by gram-negative bacterium, said kit comprising:
an amount of aminoglycoside effective for the treatment of the gram-negative bacterium; and
an amount of an aminoguanidine effective to attenuate side effects caused by the aminoglycoside.

33. A kit according to claim 32 wherein the side effect is nephrotoxicity or ototoxicity.

34. A kit according to claim 32 wherein the side effect is nephrotoxicity.

35. A kit according to claim 32 wherein the aminoguanidine is administered at 1 mg/kg/day.

36. A kit according to claim 32 wherein the aminoguanidine is administered at 150 mg to 600 mg/day.

37. A kit according to claim 32 wherein the aminoguanidine is aminoguanidine bicarbonate, aminoguanidine hydrochloride or aminoguanidine hemisulphate.

38. A kit according to claim 32 wherein the aminoguanidine is aminoguanidine bicarbonate.

39. A kit according to claim 32 wherein the aminoglycoside is selected from the group including amikacin, gentamicin, kanamycin, neomycin, netilmicin, paromomycin, streptomycin, and tobramycin

40. A kit according to claim 32 wherein the aminoglycoside is gentamicin.
